(19) United States
(12) Patent Application Publication
(10) Pub. No.: US 2016/0135862 A1
(43) Pub. Date: May 19, 2016

(54) CURVED SURGICAL TOOLS

(71) Applicant: Spinal Elements, Inc., Carlsbad, CA (US)

(72) Inventor: Eugene Shoshtaev, Del Mar, CA (US)

(21) Appl. No.: 14/942,675

(22) Filed: Nov. 16, 2015

Related U.S. Application Data
(60) Provisional application No. 62/080,881, filed on Nov. 17, 2014.

(51) Int. Cl. A61B 17/88; A61B 17/84

(52) U.S. Cl. CPC A61B 17/8872; A61B 17/84; A61B 17/8875; A61B 2017/0069

(57) ABSTRACT

Surgical tools having curved shapes for accessing a surgical site through an access channel are disclosed. The curved surgical tools can include a tip that is aligned and/or parallel with the handle to help transmit forces. Some embodiments of the surgical tool include a flexible member extending through a hollow surgical tool to transmit rotational motion from the handle to the tip. Methods of using the curved surgical tools are also disclosed.

### CROSS-REFERENCE TO RELATED APPLICATIONS

The present application claims priority benefit under 35 U.S.C. §119(e) of U.S. Provisional Application No. 62/080,881, filed Nov. 17, 2014, the content of which is incorporated by reference herein in its entirety.

### BACKGROUND

1. Field

The present application relates generally to spinal surgery, and more particularly to tools and methods used for implanting devices in the spine.

2. Background

The spinal structure can become damaged as a result of degeneration, dysfunction, disease and/or trauma. More specifically, the spine may exhibit disc collapse, abnormal curvature, asymmetrical disc space collapse, abnormal alignment of the vertebrae and/or general deformity, which may lead to imbalance and tilt in the vertebrae. This may result in nerve compression, disability and overall instability and pain. If the proper shaping and/or curvature are not present due to scoliosis, neuromuscular disease, cerebral palsy, or other disorder, it may be necessary to straighten or adjust the spine into a proper curvature with surgery to correct these spinal disorders.

The current standard of care to address the degenerative problems is to fixate the two adjacent vertebrae. Fixation is a surgical method wherein two or more vertebrae are held together by the placement of screws, rods, plates, and/or cages to stabilize the vertebrae. In many cases, the fixation is augmented by a process called fusion, whereby an implant is placed in the intervertebral space between two or more vertebrae to join the vertebrae together. By performing this surgical procedure, the relative motion between the two adjacent vertebrae is stopped, thus stopping motion of the vertebra and any potential pain generated as a result thereof.

In the surgical procedures, the implants are placed in the intervertebral space through an open procedure using retractors. The size of the incision and the amount that the tissue is retracted is preferably minimized to reduce scarring and recovery time. In addition, minimally invasive surgical techniques have been used on the spine to access the spine through small incisions. Minimally invasive techniques involve accessing the implant site through a cannula or access tube placed through a small incision to the implant site. Minimally invasive spine surgery offers multiple advantages, such as minimal tissue damage, minimal blood loss, smaller incisions and scars, minimal post-operative discomfort, and relative quick recovery time and return to normal function.

Current tools and procedures to implant devices and/or stabilize adjacent vertebrae, however, can be slow and complex. The small openings used in open procedures and the small cannulas used in minimally invasive techniques can make the implant procedure challenging. Implant tools having angled tips have been developed to help access difficult to reach angles. However, a need still exists for an easier and better apparatus and methods for stabilizing bones.

### INCORPORATION BY REFERENCE

All publications and patent applications mentioned in this specification are herein incorporated by reference to the same extent as if each individual publication or patent application was specifically and individually indicated to be incorporated by reference.

### SUMMARY

An aspect of at least one of the embodiments disclosed herein includes a surgical tool having an elongate hollow shaft with a distal end and a proximal end. The surgical tool further includes a tip at a distal end having a first longitudinal axis, a handle at the proximal end having a second longitudinal axis that is substantially parallel to the first longitudinal axis, a middle portion disposed between the tip and the handle, the middle portion having a curved shape with at least two bends. The surgical tool further includes a flexible member extending through the elongate hollow shaft with a first end coupled to the tip and a second end coupled to the handle, wherein the flexible member is configured to transmit rotational motion from the handle to the tip.

An aspect of at least one of the embodiments disclosed herein includes a surgical tool comprising a tip at a distal end, a handle at a proximal end, and a middle portion disposed between the tip and the handle, the middle portion having a curved shape with at least two bends.

In some embodiments, the tip has a longitudinal axis that is substantially coaxial with a longitudinal axis of the handle. In some embodiments, the tip has a longitudinal axis that is substantially parallel with the longitudinal axis of the handle. The tip can have a longitudinal axis that is offset from the longitudinal axis of the handle. The tip can have a longitudinal axis that is at an angle from the longitudinal axis of the handle, the angle being less than or equal to approximately 30 degrees.

In some embodiments, the surgical tool further comprises a flexible member having a first end coupled to the tip and a second end coupled to the handle, wherein the tip comprises a driver that is rotated by turning the handle. The flexible member can be a flexible rotary shaft. The flexible member can have a plurality of universal joints. The flexible member can have beveled gears. In some embodiments, the tip can have an awl or a drill.

In some embodiments, the middle portion curves in a first direction, and a second direction that is perpendicular to the first direction. In some embodiments, the middle portion is rigid and configured to transmit axial forces from the handle to the tip. The middle portion can include a first leg extending at an angle from the tip, a second leg extending at an angle from the first leg, and a third leg extending at an angle from the second leg.

In some embodiments, a width of the tip, measured as a distance perpendicular to a longitudinal axis of the first leg from a leading end of the tip to a back edge of the first leg is less than or equal to approximately 55 mm.

In some embodiments, the length of the first leg and tip, measured as a distance parallel to the longitudinal axis of the first leg from an end of the tip to the top of the first leg is less than or equal to approximately 200 mm. In some embodiments, the tip has a longitudinal axis that is offset from the longitudinal axis of the handle, the offset distance approximately equal to half the length of the first leg and tip.

An aspect of at least one of the embodiments disclosed herein includes a method of using a surgical tool, including delivering a tip of the surgical tool to an implant site, wherein the surgical tool comprises a handle at a proximal end and a middle portion disposed between the tip and the handle, the middle portion having a curved shape with at least two bends. The method further includes applying an axial force along a longitudinal axis of the handle, wherein the axial force is transmitted through the surgical tool to the tip along a longitudinal axis of the tip.

In some embodiments, the method further includes coupling a fastener to the tip of the surgical tool prior to delivering the tip to the implant site. In some embodiments, the method further includes rotating the handle to transmit a rotational torque through the surgical tool to the tip.

### BRIEF DESCRIPTION OF THE DRAWINGS

These and other features, aspects and advantages of the described embodiments are described with reference to drawings of certain preferred embodiments, which are intended to illustrate, but not to limit. It is to be understood that the attached drawings are for the purpose of illustrating concepts of the described embodiments and may not be to scale.

FIG. 1 is a perspective view of an intervertebral device implanted in a spine.

FIG. 2 is a side view showing a working channel through a patient's tissue to the intervertebral device implanted in the spine of FIG. 1.

FIG. 3 is a side view of FIG. 2 with a curved tool according to an embodiment of the present disclosure.

FIG. 4 is a side view of the curved tool of FIG. 3.

FIG. 5 is a perspective view of a curved driver tool of FIG. 3.

FIG. 6 is a cross-sectional side view of FIG. 3.

FIG. 7 is a close-up cross-sectional side view of FIG. 3.

FIG. 8 is a close-up view of the tip of the curved driver tool of FIG. 5.

FIG. 9 is a close-up view of the tip of the curved driver tool of FIG. 5 with a fastener.

FIG. 10 is a side view of a curved awl tool according to an embodiment of the present disclosure.

FIG. 11 is a close-up view of the tip of the curved awl tool of FIG. 10.

FIG. 12 is a top view of an offset curved awl tool according to an embodiment of the present disclosure.

FIG. 13 is an axial view of the offset curved awl tool of FIG. 12.

FIG. 14 is a perspective view of a curved tool according to another embodiment of the present disclosure.

FIG. 15 is a side view of the curved tool of FIG. 14.

FIG. 16 is a perspective view of a curved awl tool according to another embodiment of the present disclosure.

FIG. 17 is a side view of the curved awl tool of FIG. 16.

### DETAILED DESCRIPTION

As will be explained herein, certain embodiments of curved tools provide advantages over the prior art devices. For example, the curved tools disclosed herein can help enable easier force transmission in the axial direction for improved puncturing, drilling and fastening.

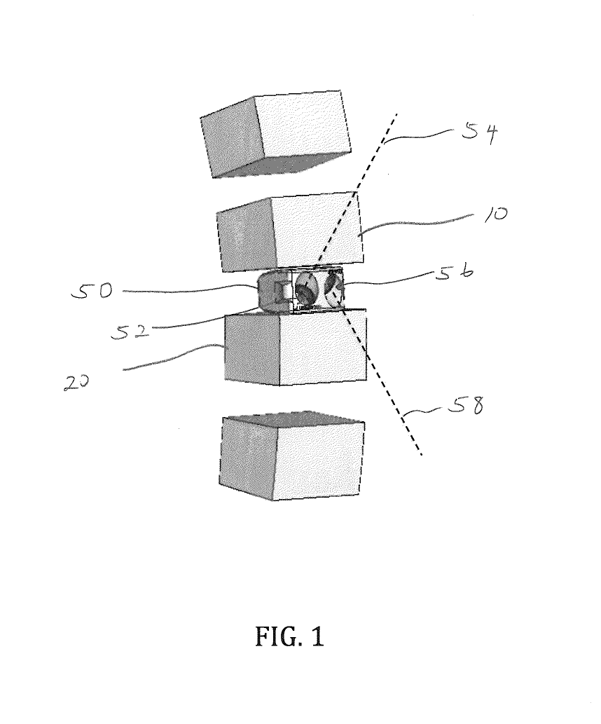

FIG. 1 illustrates an example of a device 50 implanted between a superior vertebra 10 and an inferior vertebra 20. The device 50 can have fastener holes to couple the device 50 with the superior vertebra 10 and the inferior vertebra 20. In the illustrated example, the device 50 has a first fastener hole 52 that is angled in the caudal direction such that a fastener can be inserted through the first fastener hole 52 and anchored into the inferior vertebra 20. The angle of the first fastener hole 52 is illustrated as first longitudinal axis 54. Similarly, the device 50 can have a second fastener hole 56 that is angled in the cephalic direction such that a fastener can be inserted through the second fastener hole 56 and anchored into the superior vertebra 10. The angle of the second fastener hole 54 is illustrated as second longitudinal axis 58. With continued reference to FIG. 1, the first longitudinal axis 54 and second longitudinal axis 58 are generally the directions that pilot holes are to be made in preparation for inserting the fasteners. The pilot holes can be made using an awl tool or a drill, for example.

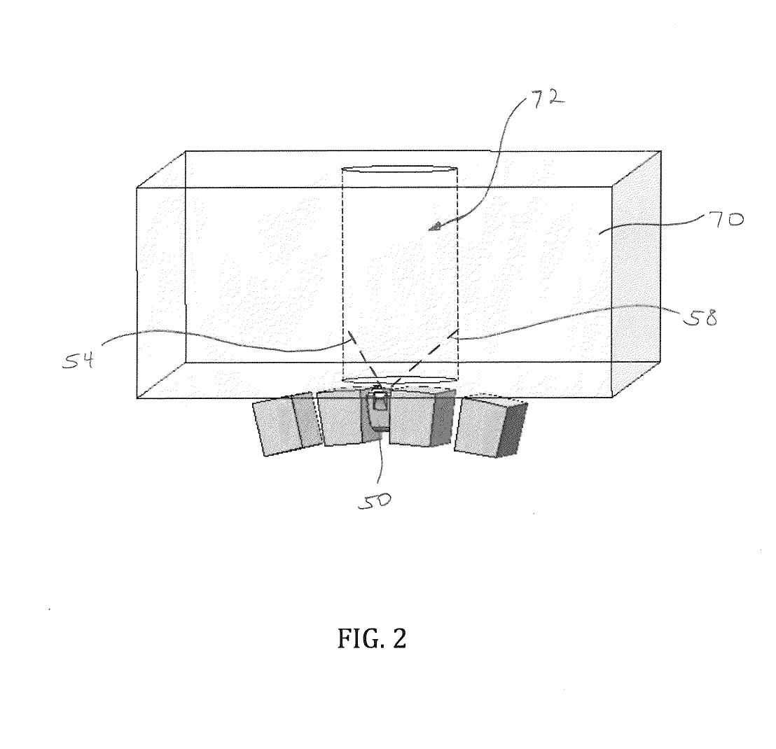

In FIG. 2, the device 50 is illustrated implanted in the spine and a representation of the patient's tissue 70 is shown above the device 50. Through the tissue 70 is an access channel 72 that can be formed using retractors or dilating cannulas, for example. The access channel 72 provides visualization and a working path to the surgical site for inserting tools. Preferably, the access channel 72 is minimal in size to minimize tissue damage and recovery time. Therefore, surgical tools can be elongate devices that are placed through the access channel to the surgical site. Oftentimes, the surgical tools have angled tips to align the tips with the angled longitudinal axes 54, 58 of the fastener holes. For example, current awls, drills and drivers are elongate tools long enough to extend through the access channel and having a handle at one end and an angled tip at the other end with the awl, drill or driver. However, the current tools can be challenging to use because it is difficult to apply forces in the axial direction of the longitudinal axes 54, 58. The tip of the current tools extend at an angle that is not aligned and not even generally aligned with the direction that the handle extends. To apply forces in the direction of the longitudinal axes 54, 58, a user must apply a transverse force on the handle or use a second tool to apply the force, which can negatively impact alignment with the fastener hole, reduce the amount of force that can be applied, increase the difficulty of use, and reduce the tactile feedback to the user.

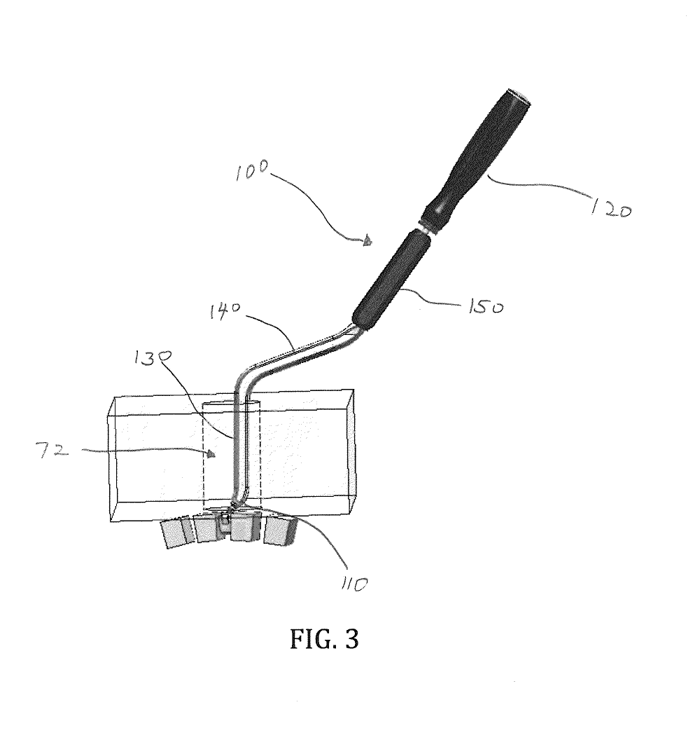

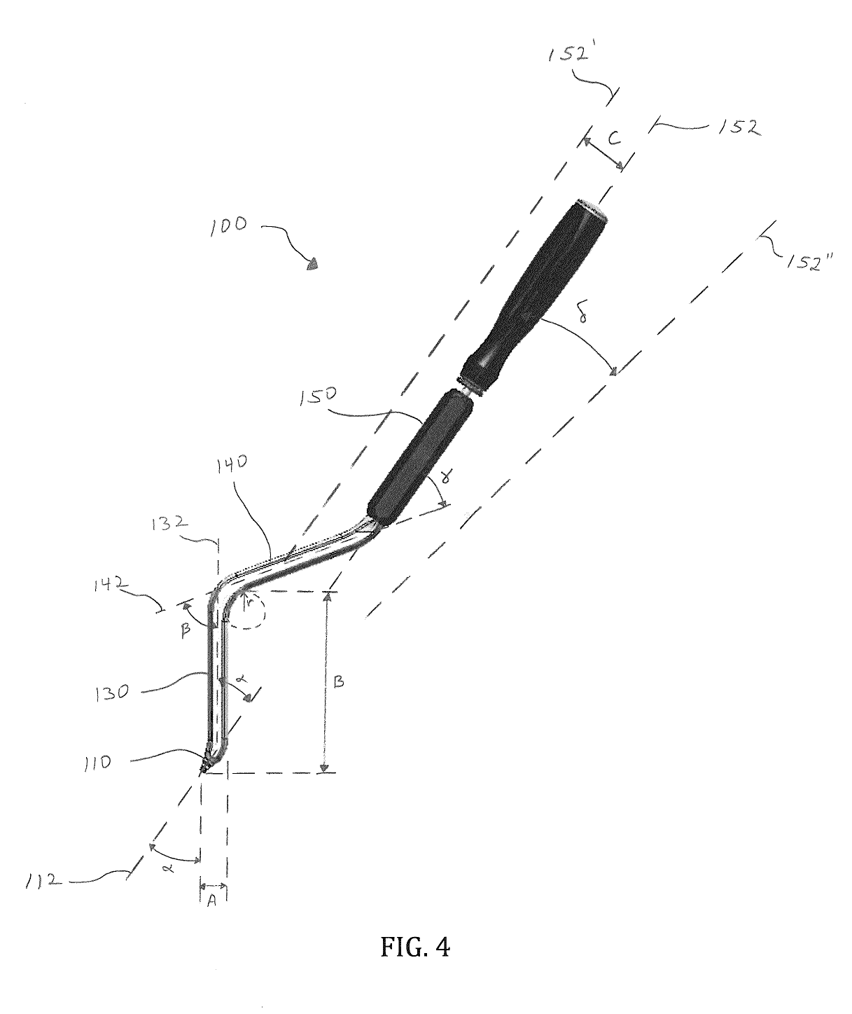

In accordance with an embodiment of the present disclosure, an improved surgical tool 100 is illustrated in FIGS. 3 and 4. The surgical tool 100 has a curved shape and when the longitudinal axis 112 of the tip 110 is aligned with one of the longitudinal axes 54, 58 of the fastener holes, the longitudinal axis of the handle 120 can also be generally aligned with the longitudinal axes 54, 58 of the fastener holes. The tip 110 can be connected to a first leg 130, wherein the longitudinal axis 112 of the tip 110 is at an angle to the longitudinal axis 132 of the first leg 130. The first leg 130 is preferably of sufficient length to extend through the length of the access channel 72, as illustrated in FIG. 3. A second leg 140 can extend from the first leg 130, wherein the longitudinal axis 132 of the first leg 130 is at an angle to the longitudinal axis 142 of the second leg 140. The second leg 140 is connected to a third leg 150, wherein the longitudinal axis 142 of the second leg 140 is at an angle to the longitudinal axis 152 of the third leg 150. The handle 120 can be attached to the third leg 150 and can be longitudinally aligned with the third leg 150. In some embodiments, the handle 120 can be at an angle to the third leg 150 such that the handle can be used as a lever for rotational motion of the tip 110.

In some embodiments, the leg lengths can be adjustable. One or more of the first leg, second leg and third leg can have a telescoping feature that enables the leg to increase and decrease in length, while still being able to transmit torque. For example, the legs can be made of two components that slideably engage with each other. A first component can have a male portion with an anti-rotational cross-section (e.g., hex shape) and a female portion with a cavity shaped to accept the male portion. The male and female portions can slide relative to each other to extend and contract, and the anti-rotational cross-section allows the leg to transmit rotational torque. Having adjustable legs can beneficially enable one surgical tool to be used for a variety of different sized patients.

Preferably, the longitudinal axis 152 of the third leg 150 is longitudinally aligned (i.e., coaxial) or substantially aligned with the longitudinal axis 112 of the tip 110. In some embodiments, the longitudinal axis 152 of the third leg 150 is generally aligned with the longitudinal axis 112 of the tip 110. In some embodiments, the longitudinal axis 152 of the third leg 150 is at an angle to the longitudinal axis 112 of the tip 110. In other embodiments, the longitudinal axis 152 of the third leg 150 is offset a distance from the longitudinal axis 112 of the tip 110.

FIG. 4 illustrates a surgical tool 100 with some dimensional references. Similar to as described above, the illustrated surgical tool 100 has a tip 110, a first leg 130, a second leg 140 and a third leg 150. The tip 110 has a longitudinal axis 112 and the first leg 130 has a longitudinal axis 132. The angle between the longitudinal axis 112 of the tip 110 and the longitudinal axis 132 of the first leg 130 is angle α. In some embodiments, the angle α is at least approximately 10 degrees and/or less than or equal to approximately 70 degrees.

The second leg 140 has a longitudinal axis 142. The angle between the longitudinal axis 132 of the first leg 130 and the longitudinal axis 142 of the second leg 140 is β. The third leg 150 has a longitudinal axis 152. The angle between the longitudinal axis 142 of the second leg 140 and the longitudinal axis 152 of the second leg 150 is γ. Preferably, the sum of the angles α and γ is approximately equal to the angle β. In other words, the longitudinal axis 112 of the tip 110 is approximately parallel with the longitudinal axis 152 of the third leg 150, as illustrated in FIG. 4. In some embodiments, the longitudinal axes 112, 152 are coaxial, which allows the ability to exert optimal axial forces at the tip 110 by applying axial loads at the handle 120.

In some embodiments, the longitudinal axis 152′ of the third leg 150 is offset from the longitudinal axis 112 of the tip 110. The longitudinal axes 152′, 112 can be offset by a distance C. The longitudinal axis 152′ can be offset to either side of longitudinal axis 112 in the view shown in FIG. 4. The maximum offset distance C can be less than or equal to approximately 25 mm. In some embodiments, the offset distance C can be less than or equal to approximately 100 mm. In some embodiments, the maximum offset distance C can be a function of the length of the first leg 130, which is labeled length B in FIG. 4. For example, the maximum offset distance C can be approximately half of length B. In other words C≈B/2.

In some embodiments, the longitudinal axis 112 of the tip 110 is at an angle to the longitudinal axis 152″ of the third leg 150, as shown by the line 152″ in FIG. 4. The angle between the longitudinal axis 152″ of third side 150 and the longitudinal axis 112 of tip 110 can be angle δ. In the view of FIG. 4, the angle can be in the clockwise direction (positive angle) or counterclockwise direction (negative angle). In some embodiments, the angle δ is at least approximately −10 degrees and/or less than or equal to approximately +10 degrees. In some embodiments, the angle δ is at least approximately −20 degrees and/or less than or equal to approximately +20 degrees. In some embodiments, the angle δ is at least approximately −30 degrees and/or less than or equal to approximately +30 degrees.

The width of the tip 110, which is the distance perpendicular to the longitudinal axis 132 of the first leg 130, measured from the leading end of the tip to the back edge of the first leg 130 is A. Preferably, the width A is minimized so that it can be operated through small incisions and cannulas, but still able to function as a driver as described below. The width A can be less than or equal to approximately 55 mm. In some embodiments, the width A is less than or equal to approximately 45 mm. In some embodiments, the width A is less than or equal to approximately 35 mm.

The length of the first leg 130 should be long enough to allow the tip 110 to reach the implant site and for the first leg 130 to extend outside of the incision, while not being too long such that the tool is unwieldy to operate. As illustrated in FIG. 4, the length B is the distance parallel to the longitudinal axis 132 of the first leg 130, measured from the end of the tip 110 to the top of the first leg 130. The top of the first leg 130 is defined as the end of the arc in the curved intersection between the first leg 130 and the second leg 140. In embodiments where the intersection between the first leg and the second leg is a sharp corner, the top of the first leg is defined as the inner corner of the intersection. Preferably, the length B is less than or equal to approximately 200 mm. In some embodiments, the length B is less than or equal to approximately 100 mm. In some embodiments, a kit can be provided to the surgeon with a plurality of different sized surgical tools. For example, the kit can include several surgical tools with first legs having length B ranging from approximately 50 mm to approximately 200 mm to accommodate patients of various sizes.

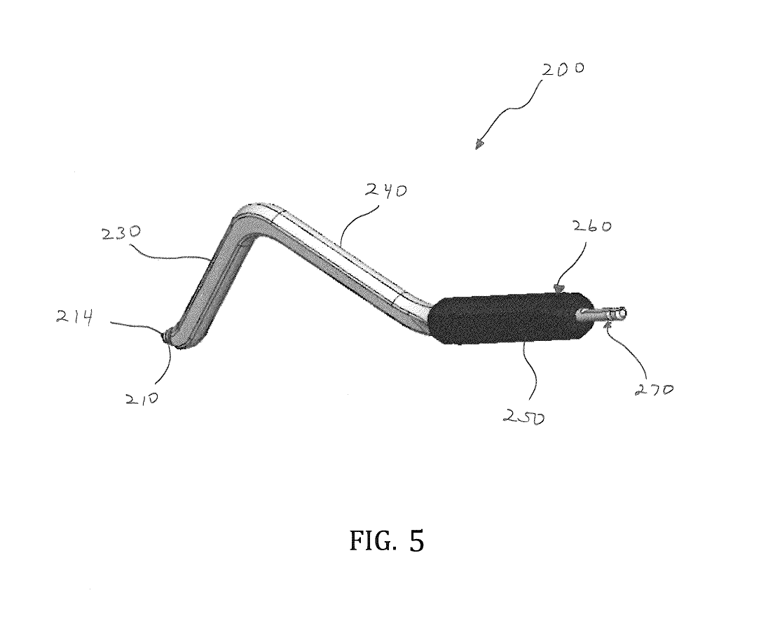

FIG. 5 illustrates a curved driver tool 200 that is configured to attach with the handle 120. Other attachments can be coupled with the handle to provide different sized drivers, awls, drills, etc. Similar to as described above for the general surgical tool, the curved driver tool 200 can have a tip 210, first leg 230, second leg 240 and third leg 250. In some embodiments, the curved driver tool 200 has a grip portion 260 around the third leg 250 for holding and stabilizing the surgical tool. The grip portion 260 can have a textured surface and/or angled shape to help the user hold onto the grip portion 260 to prevent the surgical tool from rotating during the driver actuation.

The proximal end of the curved driver tool 200 can have a coupling mechanism 270 configured to attach to the handle 120. The coupling mechanism 270 can be part of a bendable shaft or a linkage system that extends through the curved driver tool and is coupled to the driver 214 at the tip 210. In the illustrated embodiment, the coupling mechanism 270 is a shaft with a flat surface along its longitudinal length and is configured to couple with a complementary cavity in the handle 120. The flat surface provides an anti-rotational coupling with the handle 120 so that the handle 120 can be rotated about its longitudinal axis to spin the linkage system, resulting in the turning of the driver 214. Other anti-rotational configurations can be provided to attach the handle 120 and the coupling mechanism. For example, the coupling mechanism can have a polygonal cross sectional shape that is inserted into a polygonal shaped hole in the handle.

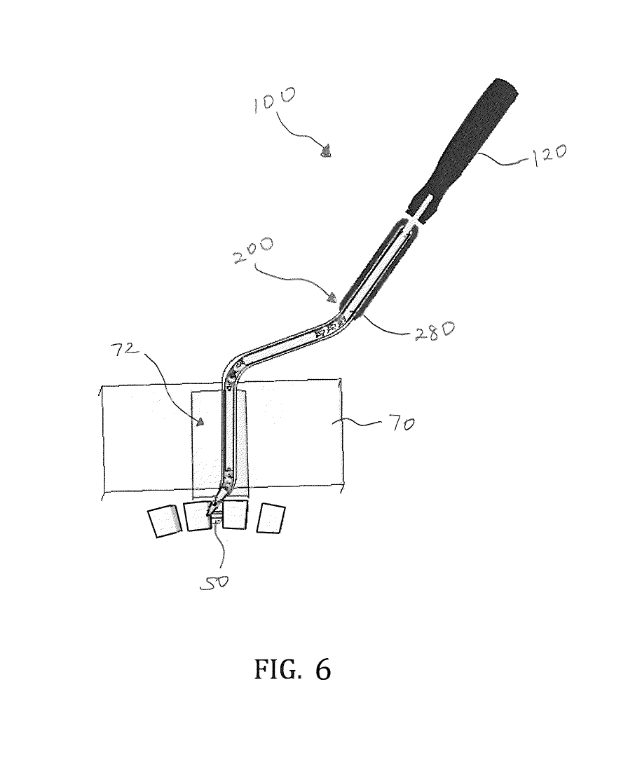

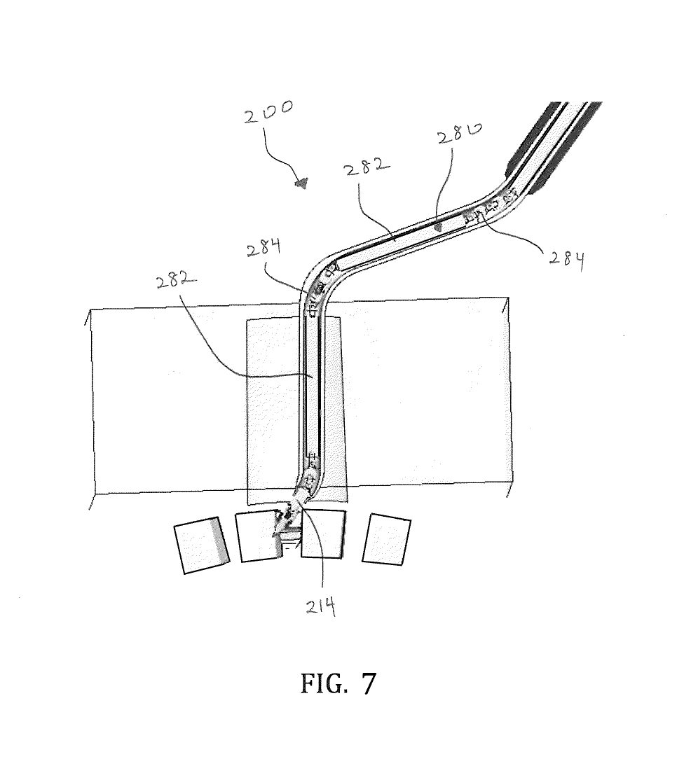

FIGS. 6 and 7 illustrate cross-sectional side views of the surgical tool 100 positioned through an access channel 72 to a device 50. The illustrated embodiment shows a curved driver tool 200 with a flexible member 280 extending through the length of the curved driver tool 200. The flexible member 280 can include rigid shaft segments that are connected with universal joints 284 disposed around the curves of the curved driver tool 200, as illustrated in the close-up view of FIG. 7. Each curve can have one, two, three or more universal joints 284 linked together depending on the size of the curve. The universal joints 284 allow the linkage member 280 to follow the curved corners while being able to transmit rotational torque through the bends of the curved driver tool 200.

In some embodiments, the flexible member 280 can have other functional designs for transmitting torque through the curves. For example, the flexible member can include a flexible rotary shaft, In other examples, the flexible member can include a wound cord, beveled gears, balled hex in socket, and the like. In some embodiments, the flexible member can be a constant velocity joint, such as a Rzeppa joint. In some embodiments, the flexible member can at least partially be made of a flexible material, such as rubber, elastic metals, or composites.

The curved driver tool 200 has a rigid shell that can transmit forces from the grip portion 260 to the tip 210. As explained above, forces can be exerted in the direction of the longitudinal axis 152 of the third leg 150 to apply the force along the longitudinal axis 112 of the tip 110. Any bending or deformation of the shell may absorb the applied force and lessen the efficiency of the transmission of forces. Also, any bending or deformation can misalign the longitudinal axes 112, 152 and affect the direction that forces are applied at the tip 110. Therefore, the shell of the curved driver tool is preferably substantially rigid so that forces are transmitted efficiently through the shell to the tip 110.

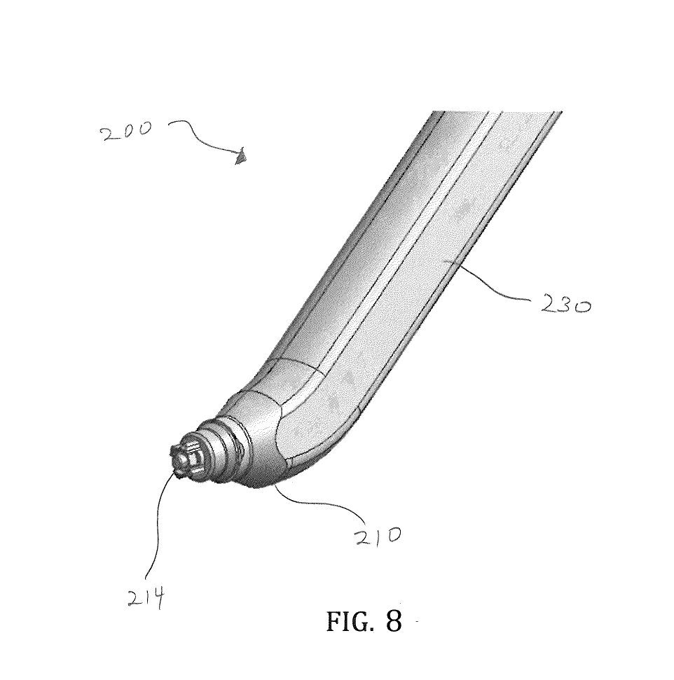

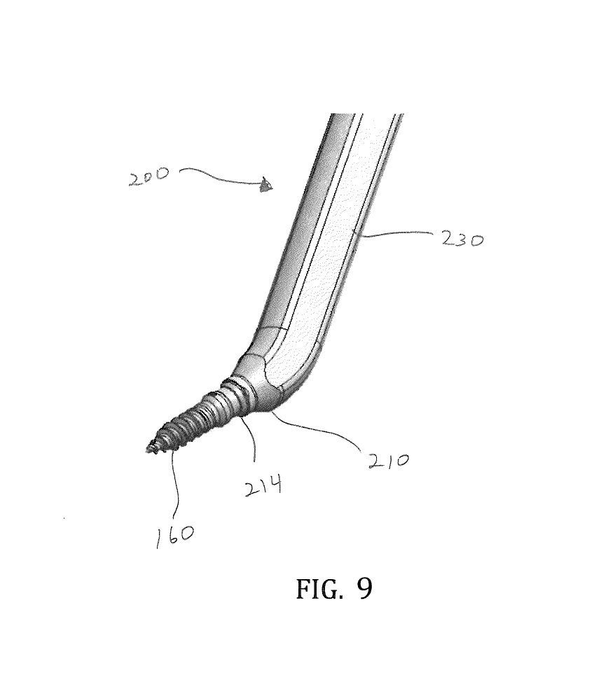

With continued reference to FIG. 7, the distal end of the curved driver tool 200 has a driver 214 that is coupled to the flexible member 280. The driver 214 can be configured to engage the head of a fastener. The driver 214 can have a cross-sectional shape that is complementary to the shape of a cavity on the head, such as a hex shape, cross shape, slot shape, Torx® shape, or other driver shapes. In the embodiment illustrated in FIG. 8, the driver 214 has a unique shape that is configured to engage special fasteners. FIG. 9 illustrates a fastener 160 coupled to the driver 214. In some embodiments, the driver is configured to attach to a drill bit, or awl, or other tool attachments. The driver can have a retaining feature to hold the drill bit, awl or other tool attachment onto the surgical tool, such as a ball and detent, hooks, or the like.

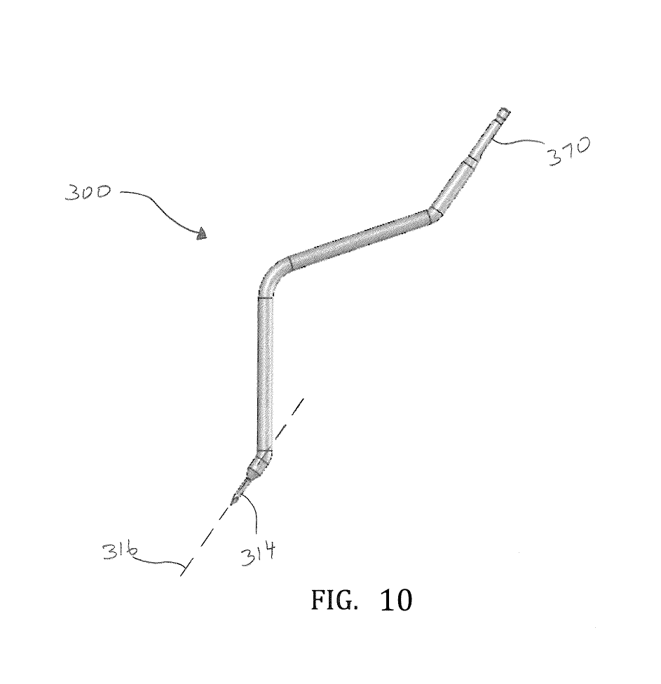

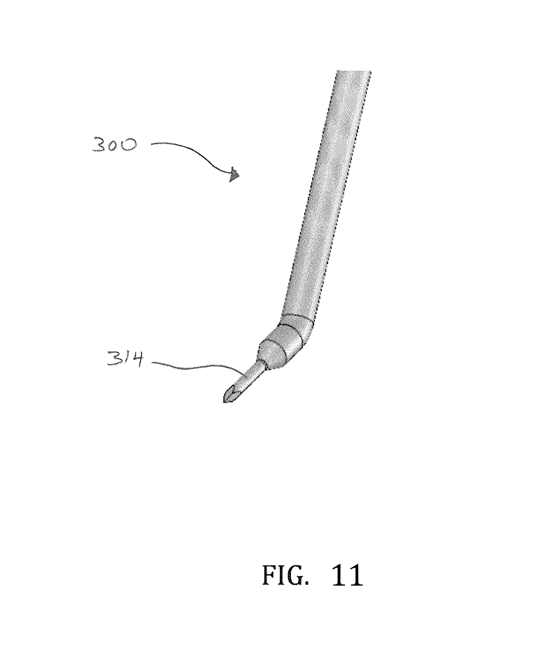

In some embodiments, the surgical tool can have a curved awl tool 300 as illustrated in FIGS. 10 and 11. The general shape of the curved awl tool 300 can be similar to as described above, with an awl 314 attached or integrally formed with the curved awl tool 300. In some embodiments, the curved awl tool 300 can be a solid shaft or a hollow shaft without a flexible member through the middle of the shaft. Instead of a flexible member to drive the rotary motion of the awl, the entire curved awl tool 300 can be rotated about the longitudinal axis 316 of the awl 314 to help drive the awl 314 into the bone.

The curved awl tool 300 can have a coupling mechanism 370 at the proximal end configured to attach to the handle 120. In the illustrated embodiment, the coupling mechanism 370 is a shaft with a flat surface along its longitudinal length and is configured to couple with a complementary cavity in the handle 120. The flat surface provides an anti-rotational coupling with the handle 120 so that the handle 120 can be rotated about its longitudinal axis to rotate the awl 314. Other anti-rotational configurations can be provided to attach the handle 120 and the coupling mechanism. For example, the coupling mechanism can have a polygonal cross sectional shape that is inserted into a polygonal shaped hole in the handle.

The curved awl tool 300 is preferably rigid to help transmit forces from the handle to the awl 314. Forces can be exerted on the handle to apply axial forces along the longitudinal axis 316 of the awl 314. Any bending or deformation of the curved awl tool 300 may absorb some of the applied force and lessen the efficiency of the transmission of forces. Also, any bending or deformation can misalign the handle 120 with the awl 314 and affect the direction that forces are applied at the awl 314. Furthermore, forces can be applied in a rotational motion to spin the awl 314 about its longitudinal axis 316. Any twisting or deformation of the curved awl tool 300 may diminish the efficiency of the transmission of forces. Therefore, the curved awl tool is preferably substantially rigid so that forces are transmitted efficiently through the tool.

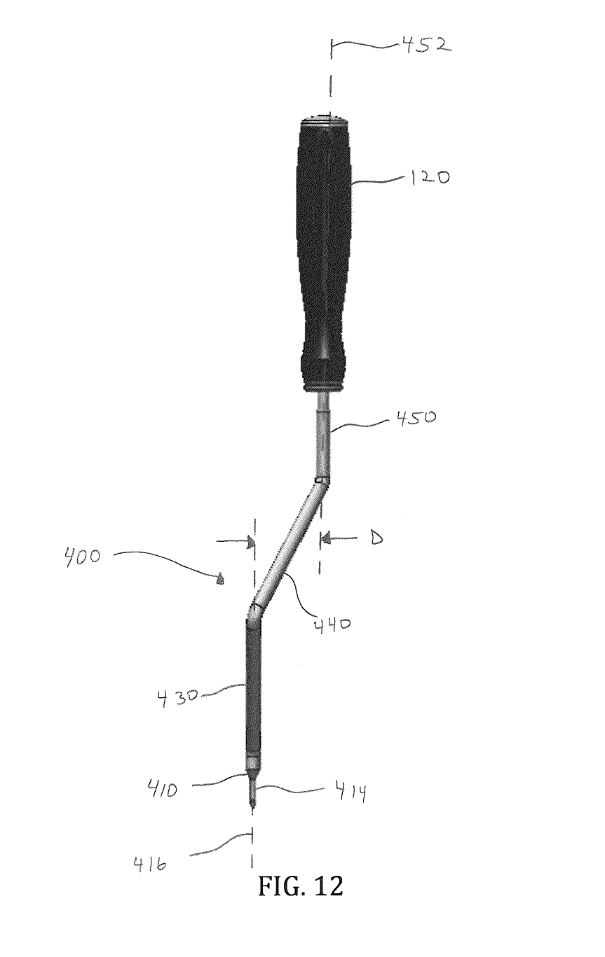

FIG. 12 illustrates a top view of a curved awl tool 400 with a lateral offset between the handle 120 and the tip 410. The top view in FIG. 12 is perpendicular to the side views shown in FIG. 4 and FIG. 10. The tip 410 has an awl 414 with a longitudinal axis 416. The curved awl tool 400 has a first leg 430, a second leg 440 and a third leg 450, the third leg 450 having a longitudinal axis 452. The lateral offset between the handle 120 and the tip 410 can be helpful for procedures where parts of the patient may interfere and not allow the tip 410 to align with the fastener holes. For example, the laterally offset curved awl tool 400 may be particularly useful for anterior cervical procedures where the patient's head, or more specifically chin, may obstruct the use of the surgical tool.

As illustrated in FIG. 12, the longitudinal axis 416 of the awl 414 can be laterally offset from the longitudinal axis 452 of the third leg 450. The distance between the longitudinal axes 416 and 452 is defined as distance D. The longitudinal axis 452 of the third leg 450 can be offset to either lateral side of longitudinal axis 416 of the awl 414. The maximum lateral offset distance D can be at most approximately 25 mm. In some embodiments, the lateral offset distance D can be greater than 25 mm. In some embodiments, the maximum lateral offset distance D can be a function of the length of the first leg 430, which is labeled length B in FIG. 4. For example, the maximum lateral offset distance D can be approximately half of length B. In other words D≈B/2. When the lateral offset distance D is zero, the longitudinal axis 416 may be coincident with longitudinal axis 452 and the handle 120 may be aligned with the tip 410, as described above.

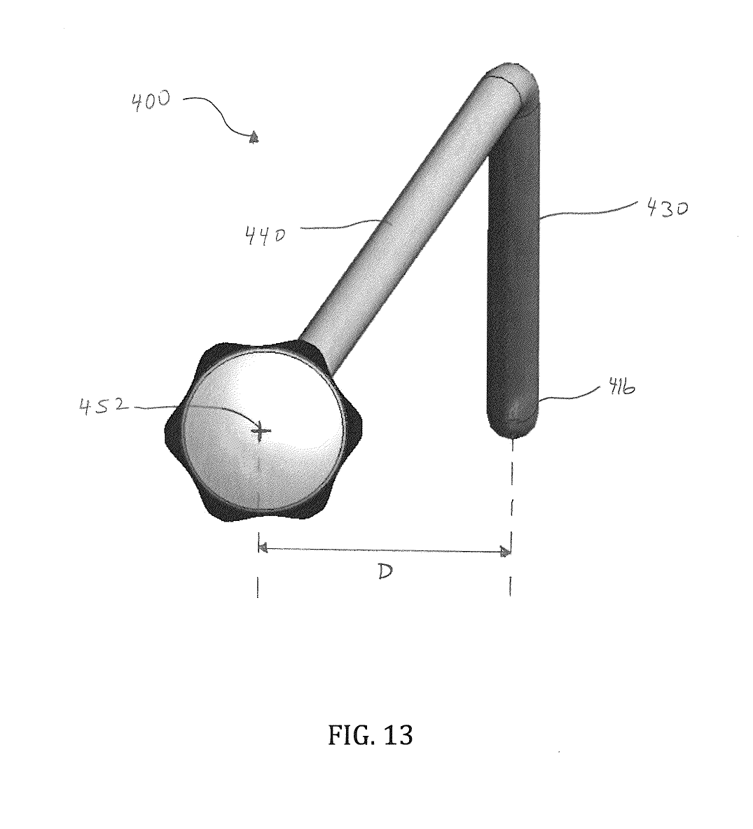

FIG. 13 is another view of the curved awl tool 400, viewed in a direction parallel with the longitudinal axes 416, 452. The distance D between the longitudinal axis 416 of the awl 414 and the longitudinal axis 452 of the third leg 450 is shown. In the illustrated embodiment, the second leg 440 extends in a lateral direction to achieve the lateral offset. In other embodiments, the first leg 430 may extend in a lateral direction instead of, or in addition to, the second leg 440 to achieve the lateral offset.

The offset of the longitudinal axis of the third leg from the longitudinal axis of the tip can be in any direction, and is not limited to only the lateral and vertical directions described above. In any direction the maximum offset distance can be a function of the length of the first leg, which is labeled length B in FIG. 4. For example, the maximum offset distance D can be approximately half of length B, or approximately B/2. Preferably, the longitudinal axes 416, 452 are substantially parallel to help apply axial forces in the direction of the awl.

In a method of using the surgical tool 100, first an access channel 72 is formed through the patient's tissue 70, for example by using retractors or cannulas, as mentioned above. A device 50 is implanted in the intervertebral space, or other surgical site. The illustrated device 50 of FIGS. 1 and 2 has angled fastener holes that require the pilot hole and fastener be driven at an angle to the direction of the access channel.

The proper sized surgical tool 100 can be selected from a kit that fits the patient's size and anatomy. A surgical tool having a length B that is longer than the depth of the access channel is selected. In situations with an adjustable surgical tool, the length of the first leg is changed so that it is slightly longer than the depth of the access channel.

The surgical tool 100 can be positioned so that the tip 110 is inserted through the access channel 72 to the device 50, as shown in FIG. 3. The surgical tool 100 can have a hole forming attachment, such as the curved awl tool illustrated in FIG. 10. The longitudinal axis 112 of the tip 110 can be aligned with the longitudinal axis of the fastener hole using direct visualization, x-ray or an alignment tool. Once the surgical tool 100 is aligned, a pushing force can be applied to the handle to transmit an axial force along the longitudinal axis 316 of the awl 314 to create a hole in the patient's bone. In some embodiments, a twisting motion can be applied to rotate the awl 314 about its longitudinal axis 316 to help form the hole. Depending on the situation, an offset curved awl tool can be used.

In some embodiments, a drill attachment can be used, where the surgical tool includes a drill bit attached to the tip of a curved driver tool. The curved driver tool can have a flexible member to rotate the drill bit, as described above. The flexible member can be rotated by the surgeon using the handle, or coupled to a powered motor to mechanically drive the drill bit. In some embodiments, the drill attachment can be a dedicated attachment with an integral drill bit at the tip.

In some embodiments, a tap attachment can be used to create threads in the bone. A tapping bit can be attached to the tip of the curved driver tool and rotated by turning the attached handle. Preferably, a powered motor is not used to prevent stripping of the threads.

Next, a fastener 160 can be inserted with the curved driver tool 200. As shown in FIG. 9, the fastener can be attached to the end of the driver. Then the surgical tool can be used to position the fasteners through the fastener holes and secured to the bone by rotating the flexible member 280. A pushing force can be exerted along the longitudinal axis of the handle 120 to transmit an axial force along the longitudinal axis of the tip 210 to help drive the fastener 160 into the bone. The flexible member 280 can be rotated by the surgeon using the handle 120, or coupled to a powered motor to mechanically drive the fastener. In some embodiments, the fasteners have self-drilling and/or self-tapping threads.

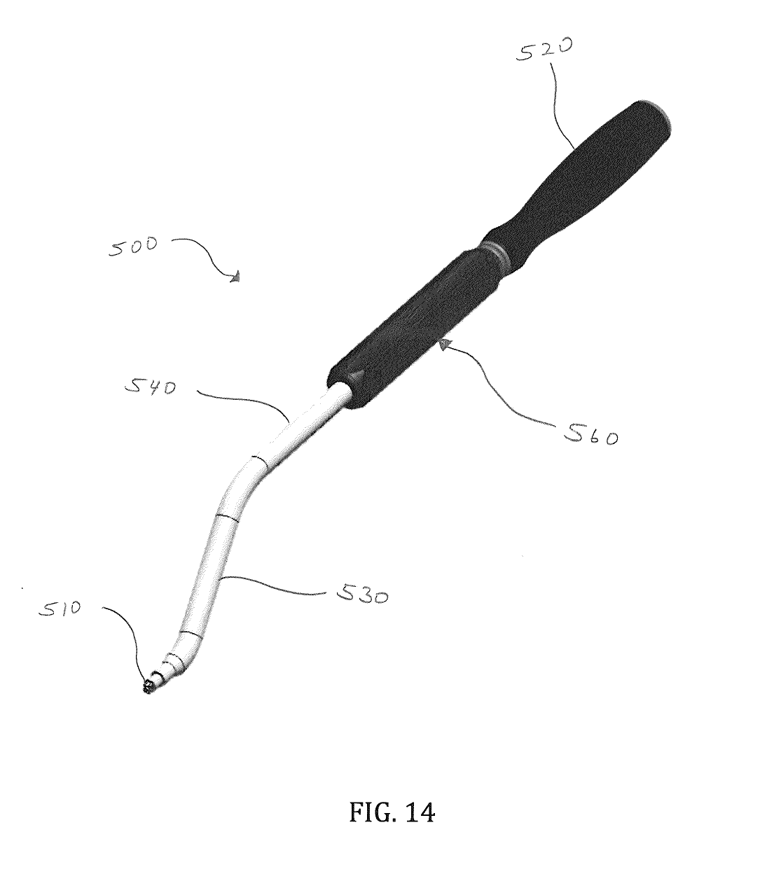

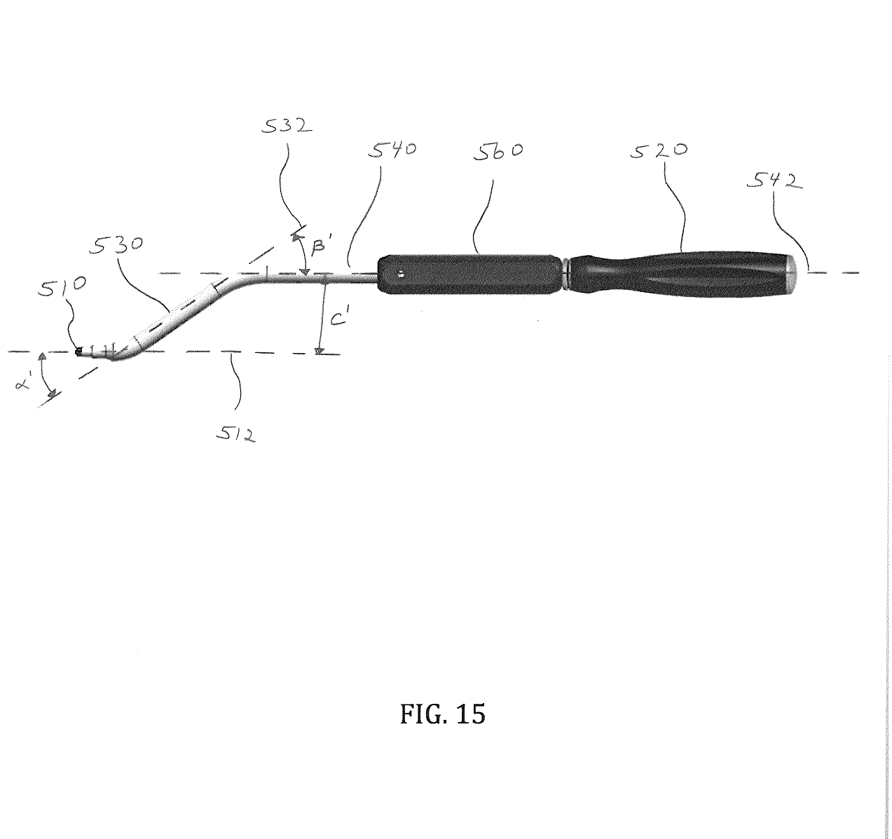

FIGS. 14 and 15 illustrate another embodiment of a surgical tool 500 having a curved shape. The surgical tool 500 can have a tip 510 that is configured to engage and drive a fastener. The tip 510 can be connected to a first leg 530, wherein the longitudinal axis 512 of the tip 510 is at an angle to the longitudinal axis 532 of the first leg 530. The first leg 530 is preferably of sufficient length to extend through the length of the access channel 72. A second leg 540 can extend from the first leg 530, wherein the longitudinal axis 532 of the first leg 530 is at an angle to the longitudinal axis 542 of the second leg 540. The second leg 540 can include a grip portion 560 for holding and stabilizing the surgical tool 500. The grip portion 560 can have a textured surface and/or angled shape to help the user hold onto the grip portion 560 and stabilize the surgical tool during the driver actuation. A handle 120 can be disposed at a proximal end of the second leg 540 and can be coupled to a flexible member that extends through the surgical tool 500 to drive the rotation of the tip 510.

In some embodiments, the lengths of the legs can be adjustable. One or more of the first leg and second leg can have a telescoping feature that enables the leg to increase and decrease in length, while still being able to transmit torque. For example, the legs can be made of two components that slideably engage with each other. A first component can have a male portion with an anti-rotational cross-section (e.g., hex shape) and a female portion with a cavity shaped to accept the male portion. The male and female portions can slide relative to each other to extend and contract, and the anti-rotational cross-section allows the leg to transmit rotational torque. Having adjustable legs can beneficially enable a surgical tool to be used for a variety of different sized patients.

With continued reference to FIG. 15, when the longitudinal axis 512 of the tip 510 is aligned with a longitudinal axis of a fastener hole, the longitudinal axis of the handle 520, which can be the same as the longitudinal axis 542 of the second leg 540, can be generally parallel with the longitudinal axis of the fastener hole. Preferably, the longitudinal axis 542 of the second leg 540 is parallel or substantially parallel with the longitudinal axis 512 of the tip 510. In some embodiments, the longitudinal axis 542 of the second leg 540 is at an angle to the longitudinal axis 512 of the tip 510.

As illustrated in FIG. 15, the angle between the longitudinal axis 512 of the tip 510 and the longitudinal axis 532 of the first leg 530 is angle α′. In some embodiments, the angle α′ is at least approximately 10 degrees and/or less than or equal to approximately 70 degrees. The second leg 140 has a longitudinal axis 542. The angle between the longitudinal axis 532 of the first leg 530 and the longitudinal axis 542 of the second leg 540 is β′.

In some embodiments, the angle α′ is the same as or approximately the same as 13′. In other words, the longitudinal axis 512 of the tip 510 can be approximately parallel with the longitudinal axis 542 of the second leg 540, as illustrated in FIG. 15. The parallel axes 512, 542 can help the user to exert forces along axis 512 at the tip 510 by applying forces at the handle 520 along axis 542.

In some embodiments, the angle α′ is different from β′ and the longitudinal axis 512 of the tip 510 is at an angle to the longitudinal axis 542 of the second leg 540. In some embodiments, the difference in angles α′, β′ is less than or equal to approximately 10 degrees. In some embodiments, the angle is less than or equal to approximately 20 degrees. In some embodiments, the angle is less than or equal to approximately 30 degrees.

In some embodiments, the longitudinal axis 542 of the second leg 540 is offset from the longitudinal axis 512 of the tip 510. The longitudinal axes 542, 512 can be offset by a distance C′. The offset distance C′ can be less than or equal to approximately 50 mm. In some embodiments, the offset distance C′ is less than or equal to approximately 150 mm.

The length of the first leg 530 can be long enough to allow the tip 510 to reach the implant site and for the first leg 530 to extend outside of the incision, while not being too long such that the tool is unwieldy to operate. Preferably, the length of the first leg 530 is less than or equal to approximately 200 mm. In some embodiments, the length of the first leg 530 is less than or equal to approximately 100 mm. In some embodiments, a kit can be provided to the surgeon with a plurality of different sized surgical tools. For example, the kit can include several surgical tools with first legs having lengths ranging from approximately 50 mm to approximately 200 mm to accommodate patients of various sizes.

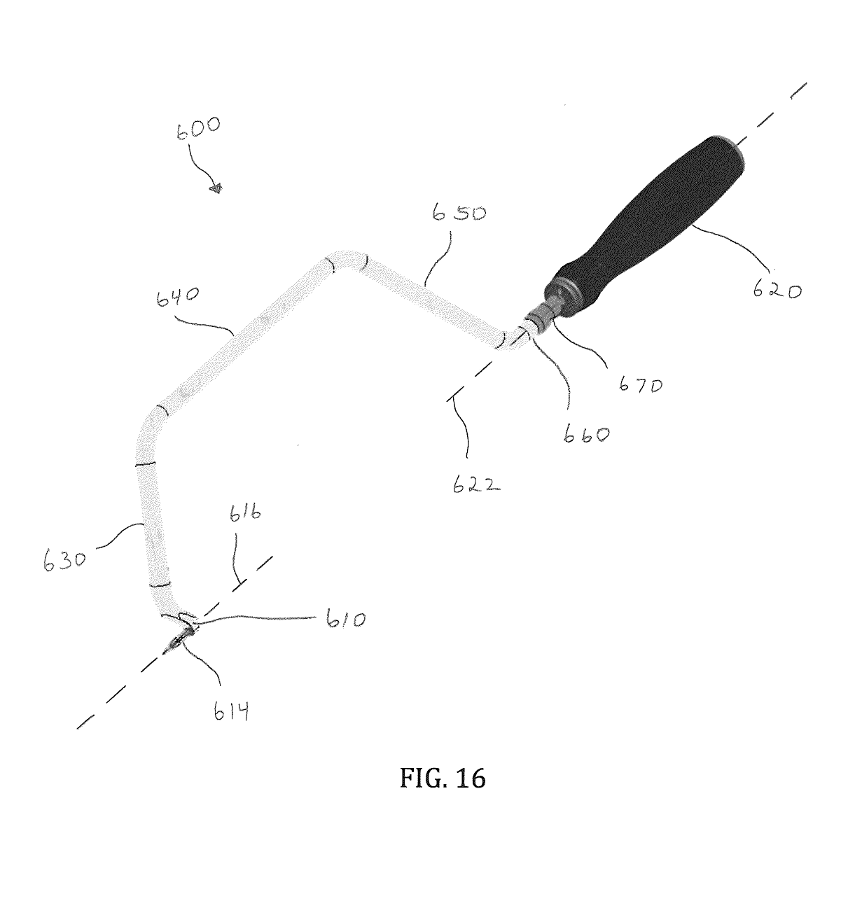

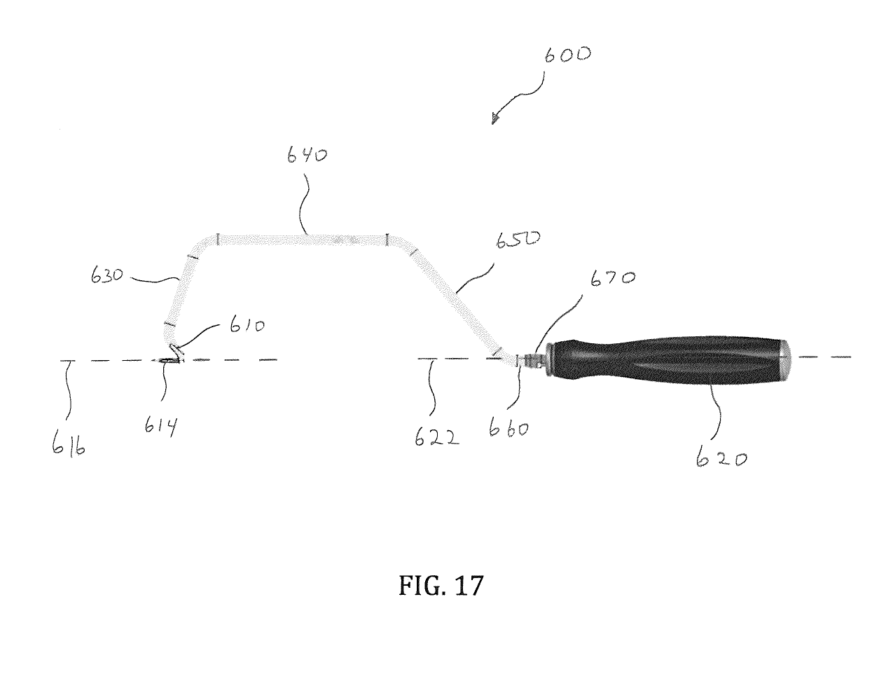

FIGS. 16 and 17 illustrate another embodiment of a curved awl tool 600 having an awl 614 attached or integrally formed with the curved awl tool 600. The curved awl tool 600 can be a solid shaft, or a hollow shaft without a flexible member through the middle of the shaft. Instead of a flexible member to drive the rotary motion of the awl, the curved awl tool 600 can be rotated about the longitudinal axis 616 of the awl 614 to help drive the awl 614 into bone.

The illustrated curved awl tool 600 includes a tip 610 at the distal end having an awl 614. The longitudinal axis of the tip 610 can be at an angle to the longitudinal axis 616 of the awl 614. The awl 614 in the illustrated embodiment is at an acute angle to the tip 610. A first leg 630 can extend at an angle to the tip 610. A second leg 640 can extend at an angle to the first leg 630. A third leg 650 can extend at an angle to the second leg 640. A fourth leg 660 can extend at an angle to the third leg 650. In some embodiments, the sum of the angles between the awl 614, tip 610, first leg 630, second leg 640, third leg 650 and fourth leg 660 is zero, wherein the longitudinal axis 616 of the awl 614 is substantially parallel or coaxial with the longitudinal axis of the fourth leg 660.

The curved awl tool 600 can have a coupling mechanism 670 at the proximal end configured to attach to a handle 620. The coupling mechanism 670 can be a shaft with a flat surface along its longitudinal length that is configured to couple with a complementary cavity in the handle 620. The flat surface provides an anti-rotational coupling with the handle 620 so that the handle 620 can be rotated about its longitudinal axis 622 to rotate the awl 614. Other anti-rotational configurations can be provided to attach the handle and the coupling mechanism. For example, the coupling mechanism can have a polygonal cross sectional shape that is inserted into a polygonal shaped hole in the handle.

In some embodiments, the longitudinal axis 622 of the handle 620 is coaxial with the longitudinal axis 616 of the awl 614. The alignment of the awl 614 and the handle 620 can help transmit longitudinal forces and rotational forces to push the awl into bone. In some embodiments, the longitudinal axis 612 of the awl 614 is parallel and offset from the longitudinal axis 622 of the handle 620. In some embodiments, the longitudinal axis 612 of the awl 614 is at an angle to the longitudinal axis 622 of the handle 620.

The curved awl tool 600 is preferably rigid to help transmit forces from the handle 620 to the awl 614. Forces can be exerted on the handle 620 to apply axial forces along the longitudinal axis 616 of the awl 614. Any bending or deformation of the curved awl tool 600 may absorb some of the applied force and lessen the efficiency of the transmission of forces. Also, any bending or deformation can misalign the handle 620 with the awl 614 and affect the direction that forces are applied at the awl 614. Furthermore, forces can be applied in a rotational motion to spin the awl 614 about its longitudinal axis 616. Any twisting or deformation of the curved awl tool 600 may diminish the efficiency of the transmission of rotational forces. Therefore, the curved awl tool is preferably substantially rigid so that forces are transmitted efficiently through the tool.

While certain embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A surgical tool comprising:
an elongate hollow shaft comprising a distal end and a proximal end;
a tip at a distal end having a first longitudinal axis;
a handle at the proximal end having a second longitudinal axis that is substantially parallel to the first longitudinal axis;
a middle portion disposed between the tip and the handle, the middle portion having a curved shape with at least two bends; and
a flexible member extending through the elongate hollow shaft and comprising a first end coupled to the tip and a second end coupled to the handle, wherein the flexible member is configured to transmit rotational motion from the handle to the tip.

2. A surgical tool comprising:
a tip at a distal end;
a handle at a proximal end; and
a middle portion disposed between the tip and the handle, the middle portion having a curved shape with at least two bends.

3. The surgical tool of claim 2, wherein the tip has a longitudinal axis that is substantially coaxial with a longitudinal axis of the handle.

4. The surgical tool of claim 2, wherein the tip has a longitudinal axis that is substantially parallel with the longitudinal axis of the handle.

5. The surgical tool of claim 2, wherein the tip has a longitudinal axis that is offset from the longitudinal axis of the handle.

6. The surgical tool of claim 2, wherein the tip has a longitudinal axis that is at an angle from the longitudinal axis of the handle, the angle being less than or equal to approximately 30 degrees.

7. The surgical tool of claim 2, further comprising a flexible member having a first end coupled to the tip and a second end coupled to the handle, wherein the tip comprises a driver that is rotated by turning the handle.

8. The surgical tool of claim 7, wherein the flexible member is a flexible rotary shaft.

9. The surgical tool of claim 7, wherein the flexible member comprises a plurality of universal joints.

10. The surgical tool of claim 7, wherein the flexible member comprises beveled gears.

11. The surgical tool of claim 2, wherein the tip comprises an awl or a drill.

12. The surgical tool of claim 2, wherein the middle portion curves in a first direction, and a second direction that is perpendicular to the first direction.

13. The surgical tool of claim 2, wherein the middle portion is rigid and configured to transmit axial forces from the handle to the tip.

14. The surgical tool of claim 2, wherein the middle portion comprises a first leg extending at an angle from the tip, a second leg extending at an angle from the first leg, and a third leg extending at an angle from the second leg.

15. The surgical tool of claim 14, wherein a width of the tip, measured as a distance perpendicular to a longitudinal axis of the first leg from a leading end of the tip to a back edge of the first leg is less than or equal to approximately 55 mm.

16. The surgical tool of claim 14, wherein the length of the first leg and tip, measured as a distance parallel to the longitudinal axis of the first leg from an end of the tip to the top of the first leg is less than or equal to approximately 200 mm.

17. The surgical tool of claim 16, wherein the tip has a longitudinal axis that is offset from the longitudinal axis of the handle, the offset distance approximately equal to half the length of the first leg and tip.

18. A method of using a surgical tool, comprising:
delivering a tip of the surgical tool to an implant site, wherein the surgical tool comprises a handle at a proximal end and a middle portion disposed between the tip and the handle, the middle portion having a curved shape with at least two bends; and
applying an axial force along a longitudinal axis of the handle;
wherein the axial force is transmitted through the surgical tool to the tip along a longitudinal axis of the tip.

19. The method of claim 18, further comprising coupling a fastener to the tip of the surgical tool prior to delivering the tip to the implant site.

20. The method of claim 18, further comprising rotating the handle to transmit a rotational torque through the surgical tool to the tip.
